(19) United States
(12) Patent Application Publication
(10) Pub. No.: US 2014/0367578 A1
(43) Pub. Date: Dec. 18, 2014

(54) X-RAY IMAGE SENSOR

(71) Applicant: Uwe Zeller, Biberach (DE)

(72) Inventor: Uwe Zeller, Biberach (DE)

(73) Assignee: FORSTGARTEN INTERNATIONAL HOLDING GMBH, St. Gallen, (CH)

(21) Appl. No.: 14/126,805

(22) PCT Filed: Jun. 18, 2012
(86) PCT No.: PCT/US12/42887

§ 371 Date: Jul. 30, 2014

Related U.S. Application Data
(60) Provisional application No. 61/497,633, filed on Jun. 16, 2011.

(51) Int. Cl. G01T 1/20; A61B 6/14; G01T 1/24

(52) U.S. Cl. CPC G01T 1/201; G01T 1/24; A61B 6/14
USPC 250/370.09

(57) ABSTRACT

An X-ray image sensor, comprising an X-ray converter layer for converting X-rays into signals received by a semiconductor detector for sampling and detecting converted X-rays as electrical signals, and a connection substrate comprising electrical connections, the X-ray converter layer bonded to a first surface of the semiconductor detector and the connection substrate arranged at a second surface of the semiconductor detector, opposite the X-ray converter layer, wherein the semiconductor detector in at least one edge portion comprises vias for through-contacting detector elements formed in or on the first surface of the semiconductor detector to the connections substrate.

### BACKGROUND

The present invention relates to X-ray imaging, including dental X-ray imaging. More specifically, the invention relates to an X-ray image sensor, comprising an X-ray converter layer for converting X-rays into signals received by a semiconductor detector for sampling and detecting converted X-rays as electrical signals, and a connection substrate comprising electrical connections, the X-ray converter layer bonded to a first surface of the semiconductor detector and the connection substrate arranged at a second surface of the semiconductor detector, opposite the X-ray converter layer.

Herein, the term “X-ray converter layer” covers any layer, in a particular plate-shaped element, which converts X-ray radiation into signals which can be received and detected by a semiconductor material, in particular into optical radiation, i.e. radiation in the visible, UV or near IR portion of the electromagnetic spectrum, irrespective of the detailed structure and composition thereof. In particular, the term covers prior art elements which consist of a fibre (or fiber) optic plate and a scintillating layer provided thereon. The term “semiconductor detector” designates any element for detecting the signals provided by the converter layer, in particular the optical radiation generated in a scintillating layer into electrical signals on a pixel-basis, i.e. comprising an array of photoelectric detector or sensor elements, respectively. Typically, the semiconductor detector converts the received signals into electrical signals. Well-known and commercially available semiconductor detectors are of the integrated silicon detector type (e.g. CCD or CMOS). The term “connection substrate” means any type of substrate comprising connections and/or electronic components which are required for operating the semiconductor detector component of the sensor and providing an internal signal processing, as far as required, irrespective of the specific type and manufacturing technology of the substrate. In particular, the term covers all types of PCBs.

An X-ray image sensor of this type is e.g. disclosed in US 2011/0013745 A1.

Such sensor comprises, as schematically illustrated in FIGS. 1A to 1D, a semiconductor detector (silicon die) 110 of basically rectangular shape, with two corners chamfered, a conventional PCB 120, and a fibre optic plate 130 with a scintillator layer 140 on that surface which is opposite to the surface where the silicon die 110 together with the PCB 120 are bonded to the fibre optic plate 130. The fiber (or fibre) optic 130 is acting as an x-ray blocking layer to absorb the x-ray intensity after the scintillating layer to prevent direct interaction of x-rays in the silicon die 110 which would cause an undesired signal thereby degrading the performance of the sensor.

Some of the prior art sensors have been built without using a fibre optic 110 by directly placing the scintillating layer onto the silicon die 110. Older prior art sensors used the silicon die itself as a converting layer, thereby accepting the weak performance efficiency of x-ray in silicon as compared to the better efficiency of newer prior art sensors, i.e. indirect working sensors using the combination of scintillating layer 140, x-ray blocking fibre optic 130 and silicon die 110 optimized for the conversion of the optical signal generated in the scintillator by the x-ray signal. Typical prior art scintillator layers are made of thallium doped caesium iodide, which has a crystal structure and a thickness of around 100 μm. Scintillators and the interface to the fibre optic (or silicon detector) are mechanically fragile. The fibre optics used in such sensors are much more mechanical stable due to their construction and their typical thickness of one to three mm. Hence, such fibre optics are inherently much less susceptible to damage induced mechanical stress.

Wire bond connections 100 are provided to connect portions or functional elements on the light-receiving surface of the silicon die 110 to connecting points on the PCB 120, which is arranged on the opposite (back or bottom) surface of the silicon die. It can be recognized that the wire bonds 100 are provided at one of the short edges of the silicon die 110 and extend over that edge to a portion of the PCB which projects over the edge of the silicon die. Whereas the fibre optic plate 130 with the scintillator layer 140 are basically congruent with the shape of the silicon die 110, they are slightly recessed with respect to the silicon die, such that the fibre optic plate does not interfere with the wire bonds 100, which are raised above the upper surface of the silicon die 110.

Furthermore, existing semiconductor detectors are such designed that all electrical connectivity, except the ground connection, must be implemented at one side of the detector, thereby substantially limiting the freedom of designing the device, i.e. the chip.

### SUMMARY OF THE INVENTION

One challenge associated with electronic intraoral X-ray systems, more specifically with sensors as described in the above U.S. patent application, is the limited space for obtaining optimized sensor signals, i.e. images of high resolution and contrast. Insofar, for such sensors it is required to optimize the ratio between that area of the sensor which is sensitive/receptive to X-ray radiation and an inactive are which is needed for electrically contacting, isolating and mechanically protecting the sensor.

It is a further challenge, to provide for a high mechanical stability of the sensor, under the constraints of limited space for the housing thereof and, more specifically, of limited thickness of the sensor as a whole.

Therefore, it is an object of the invention to provide an X-ray image sensor of the above type which combines high mechanical stability at low outer dimensions with an optimized ratio between the X-ray sensitive and the total area of the sensor and which allows enhanced freedom in the designing thereof.

This object is solved by an image sensor according to claim 1. Embodiments of the invention are subject of the dependent claims.

It is an aspect of the invention, that the semiconductor detector of the sensor in at least one edge portion, preferably at any side thereof, comprises vias for through-contacting detector elements formed in or on the first surface of the semiconductor detector to the connection substrate.

The embodiments of the invention hereinafter are disclosed for the case that a fibre optic is combined with a scintillator to form a fibre optic scintillating plate. However, those skilled in the art will appreciate that the fibre optic can be omitted or alternative types of scintillators may be used for achieving the intended improvements in respect of aspect ratios, easier and less costly production and mechanical stability.

In an embodiment of the invention, the image sensor has the overall shape of a plate, and the semiconductor detector comprises a detector plate, the X-ray converter layer comprises a fibre optic scintillating plate, and the connection substrate comprises a PCB. The plate shape of the sensor components, as well as the corresponding overall shape of the sensor in its housing is, as such, a well-known configuration but is dramatically improved in its mechanical performance by applying the inventive concept.

In an embodiment of the invention, vias and through-contacts are provided in each of the short edge portions of the semiconductor detector. Techniques for forming vias and through-contacts in a semiconductor substrate are well-known in the art, so that a detailed description of such techniques is not required.

In a further embodiment of the invention, the semiconductor detector comprises a silicon wafer portion of basically rectangular shape, in particular with at least two corners cut-off (chamfered), preferably four corners cut-off. More specifically, in this embodiment the short edge of the rectangular side of the wafer portion, as well as two or all three edges of the chamfered-corner side are provided with vias and through-contacts.

In another embodiment, the through-contacted detector elements are connected to the PCB/substrate by wire bonds. Besides wire bonding, other well-established IC connecting techniques can be used to provide the required electrical connections between the detector elements and the associated connection points on the PCB/substrate, including but not limited to ball bonding, soldering and galvanic techniques.

In another embodiment, the semiconductor detector and the PCB/substrate are geometrically similar, wherein the semiconductor detector is slightly larger than the PCB/substrate, or are basically congruent. Here, “basically congruent” means that the circumferential shape of the semiconductor detector and the PCB/substrate appear as identical, although minor local deviations may exist. In this embodiment, it is important that the PCB/substrate is not larger than the semiconductor detector, i.e. the edges of the PCB/substrate do not project over the corresponding edges of the semiconductor detector, which eliminates a drawback of prior art sensor arrangements.

In a further, closely related embodiment the X-ray converter layer, e.g. scintillating plate, and the semiconductor detector are geometrically similar, wherein the scintillating plate is slightly larger than the semiconductor detector and arranged such that none of the edges of the semiconductor detector projects over a corresponding edge of the scintillating plate. In this embodiment, the scintillating plate protects the semiconductor detector from external mechanical forces, which helps to avoid damage of the fragile and expensive semiconductor detector (specifically silicon wafer plate).

More specifically, in a further embodiment the X-ray converter layer, e.g. the scintillating plate, is self-supporting and supports and provides mechanical integrity to the semiconductor detector, which is tightly bonded to the scintillating plate and to the PCB/substrate. The tight bonding of the semiconductor detector to the scintillating plate, notwithstanding the above mentioned slightly larger dimensions of the scintillating plate, becomes possible, or is at least facilitated, by the vias and through-contacts in the edge portions of the semiconductor detector.

In a further embodiment, the X-ray converter layer, e.g. scintillating plate, the semiconductor detector and the PCB/substrate are, as an integral mechanical unit, encapsulated in a housing, the inner walls of the housing preferably tightly fitting to the outer edges of the scintillating plate. In this embodiment, the total area of the sensor, including its housing, is being minimized without increasing the risk of mechanical damage of the semiconductor detector and/or the PCB/substrate. Instead, the optimized adapted housing of this embodiment guides mechanical impacts or stress to the robust scintillating plate.

At least in some embodiments, the image sensor according to the present invention has an improved ratio between the active, i.e. X-ray sensitive area and the total sensor area, due to the replacement of standard wire connections at edges of the semiconductor detector (silicon detector) with vias and through-contacts, which makes it possible to reduce the dimensions of the PCB below those of the semiconductor detector and, at the same time, to increase the dimensions of the scintillating plate to conform to those of the semiconductor detector. Furthermore, at least in some embodiments of the invention the mechanical integrity and robustness of the image sensor are improved, due to the fact that the provision of vias and through-contacts makes it possible that the scintillating plate dominates the geometrical configuration of the sensor and at the same time provides a new dimension of mechanical integrity to the semiconductor detector and PCB, which can now tightly be bonded to the scintillating plate. Furthermore, at least in embodiments of the invention even the replacement of the mechanically fragile “classical” wire bonds, bridging the edge of the semiconductor detector down to the PCB and insofar exposed to mechanical impacts and stress, with embedded through-contacts results in improved mechanical properties and reliability of the image sensor.

### BRIEF DESCRIPTION OF THE DRAWINGS

FIGS. 1A to 1C are schematic illustrations of a plate-shaped X-ray image sensor, wherein FIG. 1A is a top view of the semiconductor detector and PCB, FIG. 1B is a side view of the semiconductor detector and PCB, and FIG. 1C is a side-view including a scintillating plate comprising a fibre optic and a scintillating layer located on the top e.g. towards the x-ray source of the fibre optic.

FIGS. 2A to 2C are schematic illustrations of a plate-shaped X-ray image sensor according to a first embodiment of the invention, wherein FIG. 2A is a top view of the semiconductor detector and PCB, FIG. 2B is a side view of the semiconductor detector and PCB, and FIG. 2C is a side-view including a scintillating plate.

FIGS. 3A and 3B are schematic illustrations of a plate-shaped X-ray image sensor according to a second embodiment of the invention, wherein FIG. 3A is a top view and FIG. 3B is a side view of the semiconductor detector and PCB.

FIGS. 4A to 4D are schematic illustrations of a plate-shaped X-ray image sensor according to a third embodiment of the invention, wherein FIG. 4A is a top view of the semiconductor detector and PCB, FIG. 4B is a side view of the semiconductor detector, FIG. 4C is a top view and FIG. 4D is a side-view of the sensor including a scintillating plate.

FIG. 5 is a schematic cross-sectional view of a further embodiment of the invention, showing a stack of scintillating plate, semiconductor detector and PCB encapsulated in a two-part plastic housing.

### DETAILED DESCRIPTION

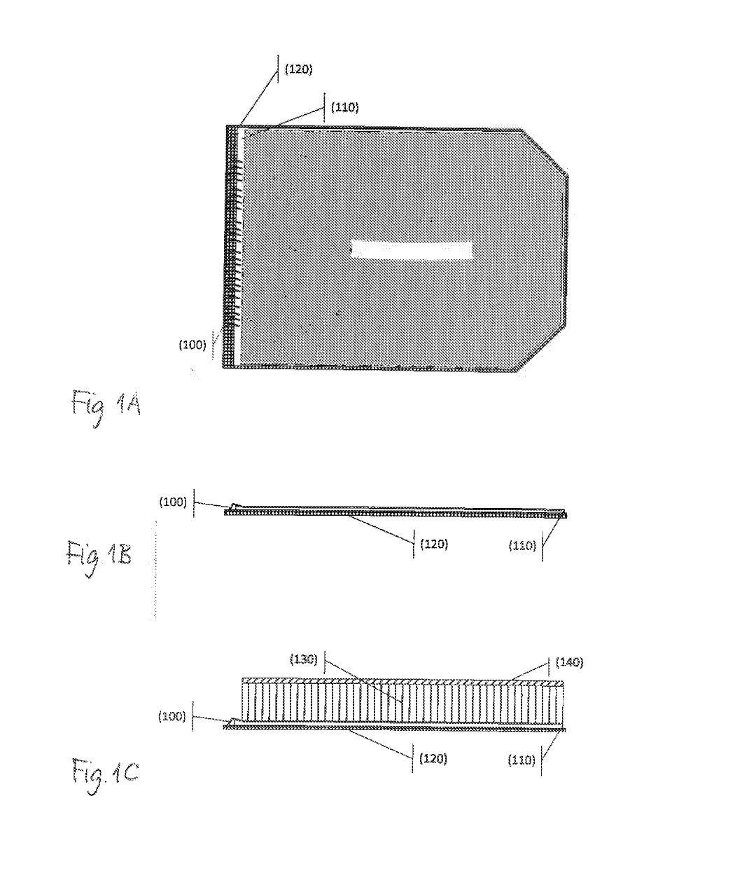

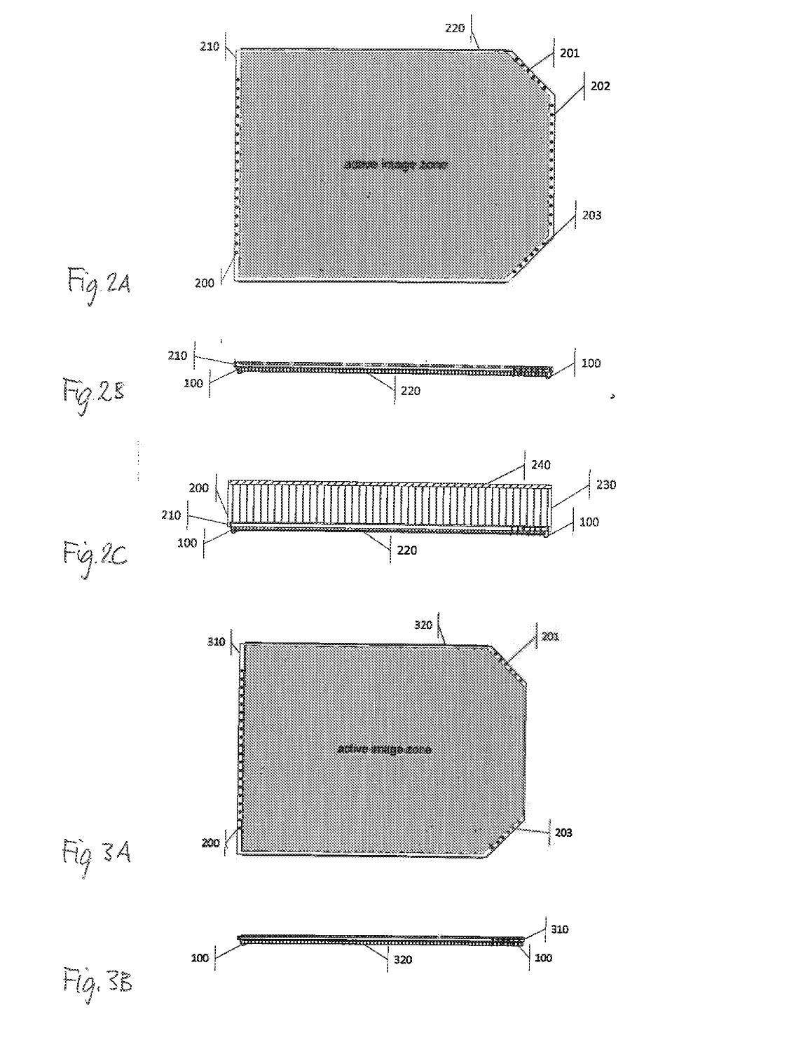

FIGS. 2A to 2C illustrate, in a similar manner as FIGS. 1A to 1C explained further above, an X-ray image sensor according to an embodiment of the invention. This sensor comprises, as schematically illustrated in FIGS. 1A to 1D, a semiconductor detector (silicon die) 210 of basically rectangular shape, with two corners chamfered, a conventional PCB 220, and a fibre optic plate 230 with a scintillator layer 240 on that surface which is opposite to the surface where the silicon die 210 together with the PCB 220 are bonded to the fibre optic plate 230.

Both at the short edge of the rectangular left portion of the silicon die 210 and in the chamfered portions and at the remaining short edge in the right portion thereof, vias 200, 201, 202 and 203, respectively, and through-contacts are provided. As best can be seen in FIG. 2B, at the bottom side of the silicon die 210, at the respective ends of the vias, wire bonds 100 are provided for contacting the ends of the through-contacts to connecting points on the PCB 220. It should be emphasized that this is just an exemplary connecting scheme, whereas further options for implementing the connections on the bottom surface of the silicon die are available and may, under certain conditions, be advantageous over the illustrated wire bonds.

What also becomes apparent from the figures, are the specific geometrical relationships between the relevant plate-shaped elements, i.e. the fibre optic/scintillator plate 230/240, the semiconductor detector 210 and the PCB 220: Different from the conventional arrangement in FIGS. 1A to 1C, the dimensions of the PCB, as compared to the semiconductor detector, are reduced, whereas the dimensions of the fibre optic/scintillator plate are increased, to make the outer edges of the latter to be at least congruent with or slightly project over the semiconductor detector. As at the light receiving surface of the semiconductor detector there are no longer any wire bonds, the fibre optic/scintillator plate 230/240 can be made as large as to cover the full adjoining surface of the semiconductor detector, and a full-surface tight bond (e.g. by means of a transparent adhesive) can be provided between them. This guarantees highest possible protection of the fragile semiconductor detector against mechanical impact or stress and high reliability, even when the sensor is applied for dental purposes, where movements, dropping, or accidental or desired biting on the sensor is likely to occur.

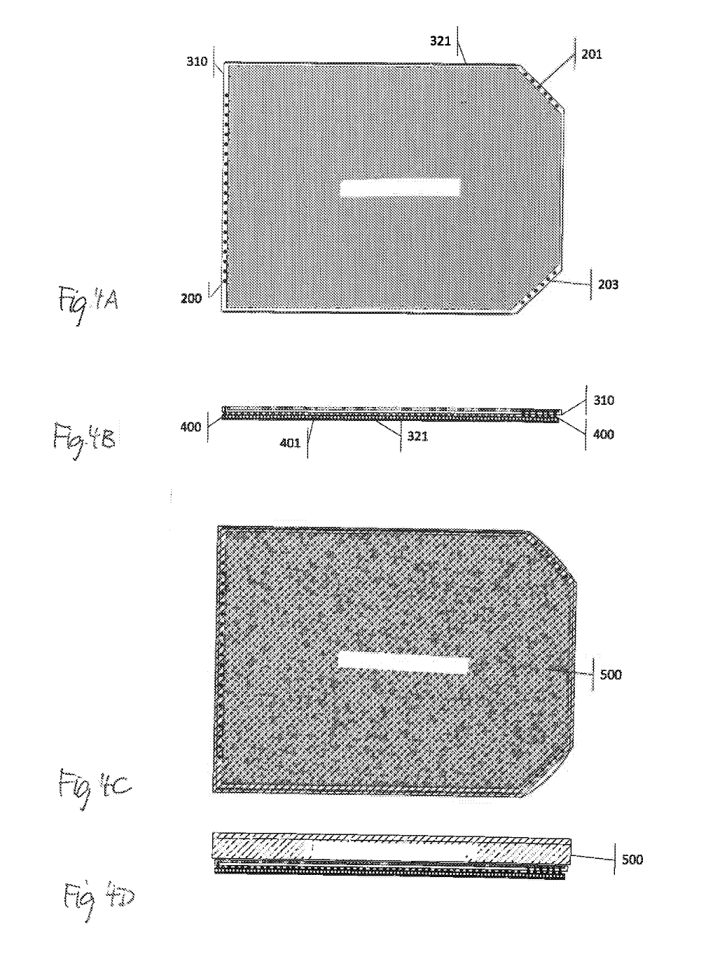

In FIGS. 3A and 3B, a modified embodiment of the invention is illustrated, wherein at the remaining shorter edge in the chamfered portion of the silicon die 310 no vias and through-contacts are provided. Except this difference, the arrangement is similar to the embodiment of FIGS. 2A to 2C.

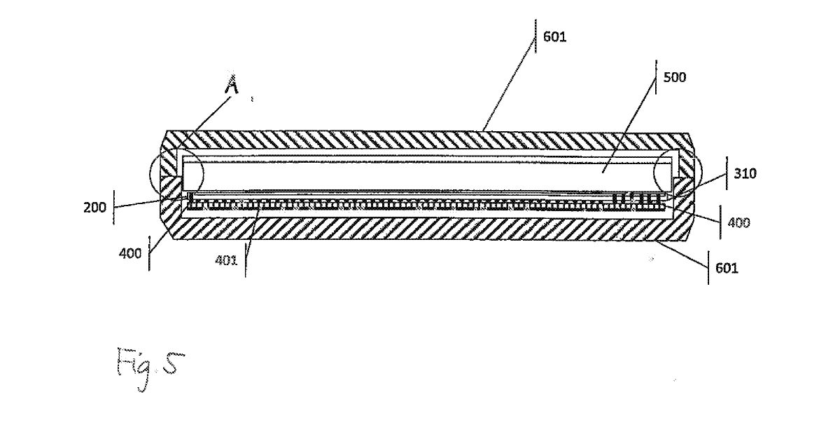

In FIGS. 4A to 4D, a further embodiment is illustrated, the semiconductor detector 310 of which is similar to the semiconductor detector 310 in FIGS. 3A and 3B. However, in the present embodiment there are no wire bond connections between the semiconductor detector 310 and the (modified) PCB 321. These are replaced with soldered connections 400, 401 which e.g. can be produced by means of a reflow soldering technique or, just to mention one of the available alternatives, by means of conductive paste screen printing. This embodiment has the additional advantage that no fragile wire bonds exist at all, not even on the bottom surface of the silicon die. In FIGS. 4C and 4D, it can be seen that on the semiconductor detector 310 a slightly larger integrated fibre optic/scintillator plate 500 is mounted, which plate has slightly convex edges in the chamfered portions of the plate stack. This provides some additional mechanical protection for these edge portions and the vias and through-contacts 201, 203 provided therein.

FIG. 5 illustrates, in a schematical cross-sectional view, the plate stack of the X-ray image sensor according to FIGS. 4C and 4D in an optimized housing 601. The two parts of the housing 601 are adapted to the circumferential shape of the fibre optic/scintillator plate 500, which is the largest part of the plate stack, such as to minimize the overall dimensions of the sensor in its housing and to guide mechanical impacts or stress to the robust fibre option/scintillator plate. In this arrangement, any mechanical contact between inner walls of the housing and the more fragile silicon die is, in normal usage of the sensor, excluded.

Various features and advantages of the invention are set forth in the following claims.

1. An X-ray image sensor, comprising:
an X-ray converter layer for converting X-rays into signals to be received by a semiconductor detector for sampling and detecting converted X-rays as electrical signals, and a connection substrate comprising electrical connections,
the X-ray converter layer bonded to a first surface of the semiconductor detector and the connection substrate arranged at a second surface of the semiconductor detector, opposite the X-ray converter layer,
wherein the semiconductor detector in at least one edge portion comprises vias for through-contacting detector elements formed in or on the first surface of the semiconductor detector to the connection substrate.

2. The image sensor of claim 1 having the overall shape of a plate and wherein the semiconductor detector comprises a detector plate, the X-ray converter layer comprises a fibre optic scintillating plate, and the connection substrate comprises a PCB.

3. The image sensor of claim 1, wherein vias and through-contacts are provided in each of the short edge portions of the semiconductor detector.

4. The image sensor of claim 1, wherein the through-contacted detector elements are connected to the connection substrate by wire bonds.

5. The image sensor of claim 1, wherein the semiconductor detector comprises a silicon wafer portion of basically rectangular shape, in particular with at least two corners cut-off.

6. The image sensor of claim 1, wherein the semiconductor detector and the connection substrate are geometrically similar, wherein the semiconductor detector is slightly larger than the connection substrate, or are basically congruent.

7. The image sensor of claim 1, wherein the X-ray converter layer and the semiconductor detector are geometrically similar, wherein the X-ray converter layer is slightly larger than the semiconductor detector and arranged such that none of the edges of the semiconductor detector projects over a corresponding edge of the X-ray converter layer.

8. The image sensor of claim 1, wherein the X-ray converter layer is self-supporting and supports and provides mechanical integrity to the semiconductor detector, which is tightly bonded to the X-ray converter layer and to the connection substrate.

9. The image sensor of claim 1, wherein the X-ray converter layer, the semiconductor detector and the connection substrate are, as an integral mechanical unit, encapsulated in a housing, the inner walls of the housing tightly fitting to the outer edges of the X-ray converter layer.

10. Use of an image sensor of claim 1 in medical imaging, in particular dental imaging.
